(19) United States
(12) Patent Application Publication
(10) Pub. No.: US 2006/0052844 A1
(43) Pub. Date: Mar. 9, 2006

(54) System and method for measuring modifying and reporting treatment compliance

(76) Inventor: Tom Newman, Newport Beach, CA (US)

Correspondence Address: DALINA LAW GROUP, P.C., 7910 IVANHOE AVE. #325, LA JOLLA, CA, 92037, US

(21) Appl. No.: 11/219,525

(22) Filed: Sep. 2, 2005

Related U.S. Application Data
(60) Provisional application No. 60/606,623, filed on Sep. 2, 2004.

(51) Int. Cl. A61N 1/18

(52) U.S. Cl. 607/67

(57) ABSTRACT

One or more embodiments of the invention are directed to a system and method for measuring, modifying, and reporting treatment compliance. The system is configured to implement one or more treatment methods on a patient and is capable of performing one or more of the various functions described herein. In one embodiment of the invention the system comprises a device configured to administer one or more of the various forms of electrical stimulation (e.g., interferential stimulation, etc . . . ) and is typically coupled with a set of electrode or other mechanism that allows electrical current from the device to flow to the proper area of treatment on a patient. The device can also contain other components configured to collect vitals or whatever other information a physician or medical professional deems necessary to make treatment decisions. The device may take various forms and can, for instance, be a part of a larger machine or incorporated into a portable device the patient can readily move to different locations where treatment might be desired. In its portable form the device may have a docking station or other mechanism to recharge the unit.

This application takes priority from United States Provisional Patent Application Ser. No. 60/606,623, filed 9/2/2004 entitled “SYSTEM AND METHOD FOR MEASURING MODIFYING AND REPORTING TREATMENT COMPLIANCE” the specification of which is hereby incorporated herein by reference.

### FIELD OF THE INVENTION

One or more embodiments of the invention relate to the field of medical devices and more particularly, but not limited to, a system and method for measuring, modifying, and reporting treatment compliance.

### BACKGROUND

There are various therapies and treatments for alleviating pain, strengthening muscles, and increasing blood circulation. One fairly common approach involves the application of electrical stimulation to a problematic area of the body. As such physicians and other health professionals regularly advise patients to make use of devices that apply one or more of the types of electrical stimulation to the body as a treatment for dealing with such ailments. Such devices are configured to regulate and manage electrical current and are typically coupled with a set of electrodes that when affixed to the patient makeup a complete system used to administer the therapy.

I. Types of Electrical Stimulation

Some examples of the different treatments that use electrical stimulation implemented in current devices follow below.

Transcutaneous Electrical Neural Stimulator (TENS)

Transcutaneous Electrical Nerve Stimulation (TENS) is a type of electrical stimulation characterized by biphasic current. Most stimulators feature adjustable settings to control amplitude (intensity) of stimulation by controlling voltage, current, and pulse width (duration) of each pulse. Electrodes are placed at specific sites on the body for treatment of pain. TENS stimulates sensory nerves to block pain signals, stimulate endorphin production to help normalize sympathetic function. Common uses of TENS include: acute and chronic pain, back and cervical muscular and disc syndromes, RSD, arthritis, shoulder syndromes, neuropathies, and many other painful conditions.

Electronic Muscle Stimulator (EMS)

Electronic Muscle Stimulators (EMS) are characterized by a low volt stimulation targeted to stimulate motor nerves to cause a muscle contraction. Contraction/relaxation of muscles has been found to effectively treat a variety of musculoskeletal and vascular conditions. EMS differs from TENS in that it is designed to stimulate muscle motor nerves, while TENS is designed to stimulate sensory nerve endings to help decrease pain. Some of the uses of EMS are as follows:

Maintaining and Increasing Range of motion: In conditions where the reduction of physiological range of motion is due to or the result of fractures with consequent immobilization, operative intervention, or arthroscopy, in shoulders, knees, and backs.

The Prevention or Retardation of muscle Disuse Atrophy: Muscle disuse atrophy is a reduction ‘in muscle contraction and size due to prolonged impairment or joint immobility from surgery, injury or disease. The use of electrical stimulation to contract the muscles builds and strengthens the muscles, assisting in prevention of disuse atrophy.

Relaxation of muscle Spasms: Muscle spasms and cramping often occur in areas of localized pain and tenderness. Stimulation is used to fatigue the “spastic” muscle. Muscle Reeducation: Evidence has shown that a combination of both exercise and electrical stimulation is far superior in strengthening atrophied muscles.

Increased Local Blood Circulation: Rhythmic muscle contraction helps improve blood circulation, thereby aiding in the reduction of localized swelling and tenderness. Immediate Post-surgical Stimulation of Calf Muscles to Prevent Venous Thrombosis: The use of EMS to increase blood circulation assists in the prevention of venous thrombosis.

Interferential Stimulator (IF)

Interferential electrical stimulation's is considered by many to be a unique way of effectively delivering therapeutic frequencies to tissue. Conventional TENS and Neuromuscular stimulators use discrete electrical pulses delivered at low frequencies of 2-160 Hz per second. However, Interferential stimulators use a fixed carrier frequency of 4,000 Hz per second and also a second adjustable frequency of 4,001-4,400 Hz per second. When the fixed and adjustable frequencies combine (heterodyne), they produce the desired signal frequency (Interference frequency). Interferential stimulation is concentrated at the point of intersection between the electrodes. This concentration occurs deep in the tissues as well as at the surface of the skin. Conventional TENS and Neuromuscular stimulators deliver most of the stimulation directly under the electrodes. Thus, with Interferential Stimulators, current perfuses to greater depths and over a larger volume of tissue than other forms of electrical therapy.

When current is applied to the skin, capacitive skin resistance decreases as pulse frequency increases.’ For example, at a frequency of 4,000 Hz (Interferential unit) capacitive skin resistance is eighty (80) times lower than with a frequency of 50 Hz (in the TENS range). Thus, Interferential current crosses the skin with greater ease and with less stimulation of cutaneous nociceptors allowing greater patient comfort during electrical stimulation. In addition, because medium-frequency (Interferential) current is tolerated better by the skin, the dosage can be increased, thus improving the ability of the Interferential current to permeate tissues and allowing easier access to deep structures. This explains why Interferential current may be most suitable for treating patients with deep pain, for promoting osteogenesis in delayed and nonunion fractures and in pseudothrosis, for stimulating deep skeletal muscle to augment the muscle pump mechanism in venous insufficiency, and for depressing the activity of certain cervical and lumbosacral sympathetic ganglia in patients with increased arterial constrictor tone. increased circulation. Common uses: Pre and post-orthopedic surgery, joint injury syndrome, cumulative trauma disorders, increasing circulation and pain control of various origins.

High Voltage Pulsed Galvanic Stimulator (HVPGS)

High-voltage pulsed galvanic stimulation (HVPGS) is gaining widespread use for wound healing, edema reduction and pain relief Carpal Tunnel Syndrome and Diabetic Foot are two major areas of use. Devices in this class are characterized by a unique twin-peak monophasic waveform with very short pulse duration (microseconds) and a therapeutic voltage greater than 100 volts. The combination of very short pulse duration and high peak current, yet low total current per second (Microcurrent) allows relatively comfortable stimulation. Furthermore, this combination provides an efficient means of exciting sensory, motor and pain-conducting nerve fibers. Perceptual discrimination of those responses is relatively easy to achieve and thus its clinical versatility.

Microcurrent Electrical Neuromuscular Stimulator (MENS)

The newest units use a very low voltage current, usually between 1 uA and 1000 uA. A microamp (uA) is 1/1000 of a milliamp (mA), so 1000 uA equals 1 mA. Most TENS devices have a milliamplitude of 1-80 mA.

It has been found that ATP (Adenosine Triphosphate) in the cell helps promote protein synthesis and healing. The lack of ATP due to trauma of the tissue results in the decreased production of sodium and an increase in metabolic wastes, which is perceived as pain. The use of Microcurrent at an injured area helps realign the body's electrical current, increase the production of ATP, resulting in increased healing and recovery, as well as blocking the pain that is perceived.

Where TENS is used to hide pain, Microcurrent, because of its close proximity of our own body's current, is thought to work on a more cellular level. It has been theorized that healthy tissue is the result of the direct flow of electrical current throughout the body. Electrical balance is disrupted when the body is injured at a particular site, causing the electrical current to change course. The use of Microcurrent over the injured site is thought to realign this flow, thus aid in tissue repair. It's been found that ATP (Adenosine Triphosphate) in the cell helps to promote protein synthesis and healing. The lack of ATP due to trauma of the tissue results in the decreased production of sodium and an increase in metabolic wastes, which is perceived as pain. The use of Microcurrent at an injured area helps to realign the body's electrical current, increase the production of ATP, resulting in increased healing and recovery, as well as blocking the pain that is perceived.

Microcurrent Stimulator (MC)

Microcurrent is used for the relief of pain, because of it's close proximity to our own body's current, and is thought to work on a more cellular level. Microcurrent is measured in MicroAmps, millionths of an ampere. Current levels that seem to be most effective in helping tissue heal range from 20 to 500 MicroAmps.

It is theorized that health tissue is the result of direct flow of electrical current throughout our body. Electrical balance is disrupted when the body is injured at a particular site, causing the electrical current to change course. The use of Microcurrent over the injured site is thought to realign this flow. Thus aid in tissue repair.

II. Compliance and Reporting Issues

As is evident from the above passage there are a number of different nuances and subtleties involved in electing which form of treatment/stimulation is proper to administer. Hence what type of stimulation to use, the frequency of such stimulation, and how to best vary between one or more different types of stimulation are all decisions best made under the close supervision of a trained professional. What often occurs however is that patients must rely upon their own self assessment in order to determine how to modify their therapy. This happens because stimulation devices are sometimes provided or recommended to the patient based on an initial visit with a physician or therapist and although the patient may be shown how to administer the stimulation, the patient is unlikely to know all of the various circumstances that may dictate a change in treatment. For instance, the frequency or types of stimulation applied may vary as time progresses. Moreover, as time passes the circumstances present at the initial visit may change and thereby cause the treatment protocol to require modification. Without regular visits to the medical professional charged with managing the stimulation most patients do not know how or when to modify their own therapy and there is currently no way from medical professional to monitor the patients treatment regime to ensure treatment compliance and then make whatever modifications might be required when circumstances change.

An additional problem faced by many of the professionals that advise patients to undergo various forms of electrical stimulation for treatment is that unless patients visit their office it is often difficult for the doctors to obtain reimbursement for any time spent determining if changes should be made to the patients therapy. Brief telephone conversations with a patient or an exchange of email are not usually compensated (e.g., by an insurance company) in the same way that an in-office dialog with the patient which involves a review of the patients medical chart.

Electrical stimulation is sometimes part of an ongoing treatment to injuries obtained on the job. When such injuries occur there is a need to determine patient compliance with the treatment given. Whether that treatment is electrotherapy, CPM's, breathing assistance for sleep apnea sufferers or diet and exercise regimes, studies show that patients that do not accept and comply with their Physician prescribed therapies in the first two weeks probably never will. Non-compliance leads to heavy increases in health costs:
Extended rehabilitation costs,
Rental dollars wasted on un-uses rental equipment,
Unnecessary extended burden on the healthcare system,
Exacerbation of the injury or illness,
Additional, collateral injury or illness due to inability to heal the original condition.

Because of the problems set forth above there is a need for a device that enables medical professionals to obtain better information about a patient's treatment regime while reducing overall treatments costs and providing increased revenue opportunities to the medical professional offering the service. Such a device would also provide benefits in that it would enable insurance companies to determine if reimbursement is proper based on the type of treatment administered. There is currently no device that solves the numerous problems identified above.

### SUMMARY OF THE INVENTION

One or more embodiments of the invention are directed to a system and method for measuring, modifying, and reporting treatment compliance. The system is configured to implement one or more treatment methods on a patient and is capable of performing one or more of the various functions described herein.

In one embodiment of the invention the system comprises a device configured to administer one or more of the various forms of electrical stimulation (e.g., interferential stimulation, etc . . . ) and is typically coupled with a set of electrode or other mechanism that allows electrical current from the device to flow to the proper area of treatment on a patient. The device can also contain other components configured to collect vitals or whatever other information a physician or medical professional deems necessary to make treatment decisions. The device may take various forms and can, for instance, be a part of a larger machine or incorporated into a portable device the patient can readily move to different locations where treatment might be desired. In its portable form the device may have a docking station or other mechanism to recharge the unit.

One aspect of the invention contemplates the inclusion of a communication component configured to allow various forms of the device to connect with a management component. The management component comprises a computer or other specialized machine configured to obtain historical data from the various devices and thereby enable a medical professional to monitor patient compliance and handle interactions with the patient. The person charged with management of treatments administered via the device can also use the management component to modify the therapy based on reported compliance data and/or interact with the patient.

The communication component referenced herein may comprise a modem, computer network or any other interconnection fabric (wireless or wired) able to connect one or more of the devices with one or more of the management components. These communication components may be incorporated into the devices themselves, incorporated into the docking station or recharge unit, or part of a stand-alone component such as a cell phone or other communication device.

When a decision is made to provide patients with a device for treatment the initial protocol or form of treatment can be entered by the medical professional responsible for oversight of the treatment. The patient is then provided with a portable device that can be taken to a location of the patient's choosing for administration of the treatment protocol. Using the communication component, the device periodically or regularly reports the patients patterns of use back to the management component. Based on these reports the operator of the management component can opt to modifying the therapy and/or simply store the reports to prove patient compliance with the treatment. Such proof enables the medical professional to know when a patient is non-compliant and then follow-up or take whatever next steps are required to attempt to achieve compliance. In some instance, the management component may, for instance, be configured to automatically provide the patient with a reminder (e.g., via email or phone) that a treatment was missed. The management component can be programmed to have various levels of non-compliance and take various actions at different levels. In the case of substantial non-compliance, for example, the system may report to a physician or insurance company who can then take whatever actions are desired (e.g., a phone call, letter, etc . . . ).

The historical use data collected from the devices also serves as a rich source for data mining to determine treatment effectiveness or derived other useful information. Although patient specific data must typically be removed to perform such analysis (particularly in the cases where the data or results of such analysis might be sold) mining the report data serves as a potential source of valuable information.

The management component may also contain an operator interface that enables a physician or other medical professional to review patient usage patterns and collect other information (e.g., vitals) in order to determine the effectiveness of the treatment regime. Based on the information obtained via this operator interface or based on the operators own personal knowledge, the operator may then modify the treatment protocol (e.g., by changing the duration or treatment, type of treatment or whatever other variables are considered pertinent). Upon receipt of an authorized modification command, the management component communication with the device via the communication component and initiates a change in the treatment protocol. Embodiments of the invention also contemplate using the communication component for messages and or data between the operator and patient.

Having the ability to set an initial treatment protocol (which can be varied by the patient within a range if such permission is granted by the medical professional responsible for treatment), remotely measure effectiveness and determine if the patient is complying with that treatment, and remotely modify the treatment based on reported treatment data provides a number of benefits in efficiency and thereby leads to increased revenue opportunities.

Insurance companies are sometimes reluctant to pay for claims relating to an ongoing treatment unless it is feasible to provide proof the treatment is truly ongoing. The device described herein, when combined with the management component provides a mechanism for not proving compliance with the treatment and can thereby lead to a higher profitability for those who receive payment from the insurance company. From the insurance companies perspective one or more aspects of the invention described herein provide a way to minimize fraud by guaranteeing that only compliant claims are paid. In instances where the patient is non-compliant, the physician is given a mechanism to seek further revenue by encouraging patient compliance.

One benefit of having a device and management component configured to communication is that it provides physicians with an efficient conduit to interact with their patient. In one or more embodiments of the invention, the management component is designed to interface with a patient's medical record and thereby given the physician enough information to conduct a remote patient consultation. For instance, when reviewing a patient's compliance report along with the patient's medical record the physician or other medical professional may use the conduit on the device to ask the patient questions and or obtain further information about the patients vitals and/or general condition. As such the system described herein enables an efficient doctor patient visit to occur when both doctor and patient are in separate locations. In one or more embodiments of the invention the device is programmed upon receipt of correspondence from the operator to only initiate treatment once the patient has responded in the requested manner to the correspondence.

### BRIEF DESCRIPTION OF THE DRAWINGS

FIG. 1A is the left third of a flow diagram for the user interface.

FIG. 1B is the center third of a flow diagram for the user interface.

FIG. 1C is the right third of a flow diagram for the user interface.

FIG. 2A is a portion of the hardware schematic for the device.

FIG. 2B is a portion of the hardware schematic for the device.

FIG. 2C is a portion of the hardware schematic for the device.

FIG. 2D is a portion of the hardware schematic for the device.

FIG. 2E is a portion of the hardware schematic for the device.

FIG. 2F is a portion of the hardware schematic for the device.

FIG. 2G is a portion of the hardware schematic for the device.

FIG. 2H is a portion of the hardware schematic for the device.

FIG. 21 is a portion of the hardware schematic for the device.

FIG. 3A is a portion of the hardware schematic for the device cradle.

FIG. 3B is a portion of the hardware schematic for the device cradle.

FIG. 3C is a portion of the hardware schematic for the device cradle.

FIG. 3D is a portion of the hardware schematic for the device cradle.

FIG. 3E is a portion of the hardware schematic for the device cradle.

FIG. 4 is a flow chart illustrating an embodiment of a method for measuring, modifying and reporting treatment compliance.

### DETAILED DESCRIPTION OF THE INVENTION

A system and method for measuring, modifying and reporting treatment compliance will now be described. In the following exemplary description numerous specific details are set forth in order to provide a more thorough understanding of embodiments of the invention. It will be apparent, however, to an artisan of ordinary skill that the present invention may be practiced without incorporating all aspects of the specific details described herein. In other instances, specific features, quantities, or measurements well known to those of ordinary skill in the art have not been described in detail so as not to obscure the invention. Readers should note that although examples of the invention are set forth herein, the claims, and the fill scope of any equivalents, are what define the metes and bounds of the invention.

The system is configured to implement one or more treatment methods on a patient and is capable of performing one or more of the various functions described herein. In one embodiment of the invention the system comprises a device configured to administer one or more of the various forms of electrical stimulation (e.g., interferential stimulation, etc . . . ) and is typically coupled with a set of electrode or other mechanism that allows electrical current from the device to flow to the proper area of treatment on a patient. The device can also contain other components configured to collect vitals or whatever other information a physician or medical professional deems necessary to make treatment decisions. The device may take various forms and can, for instance, be a part of a larger machine or incorporated into a portable device the patient can readily move to different locations where treatment might be desired. In its portable form the device may have a docking station or other mechanism to recharge the unit.

One or more embodiments of the invention comprise a communication component configured to allow various forms of the device to connect with a management component. The management component comprises a computer or other specialized machine configured to obtain historical data from the various devices and thereby enable a medical professional to monitor patient compliance and handle interactions with the patient. The person charged with management of treatments administered via the device can also use the management component to modify the therapy based on reported compliance data and/or interact with the patient.

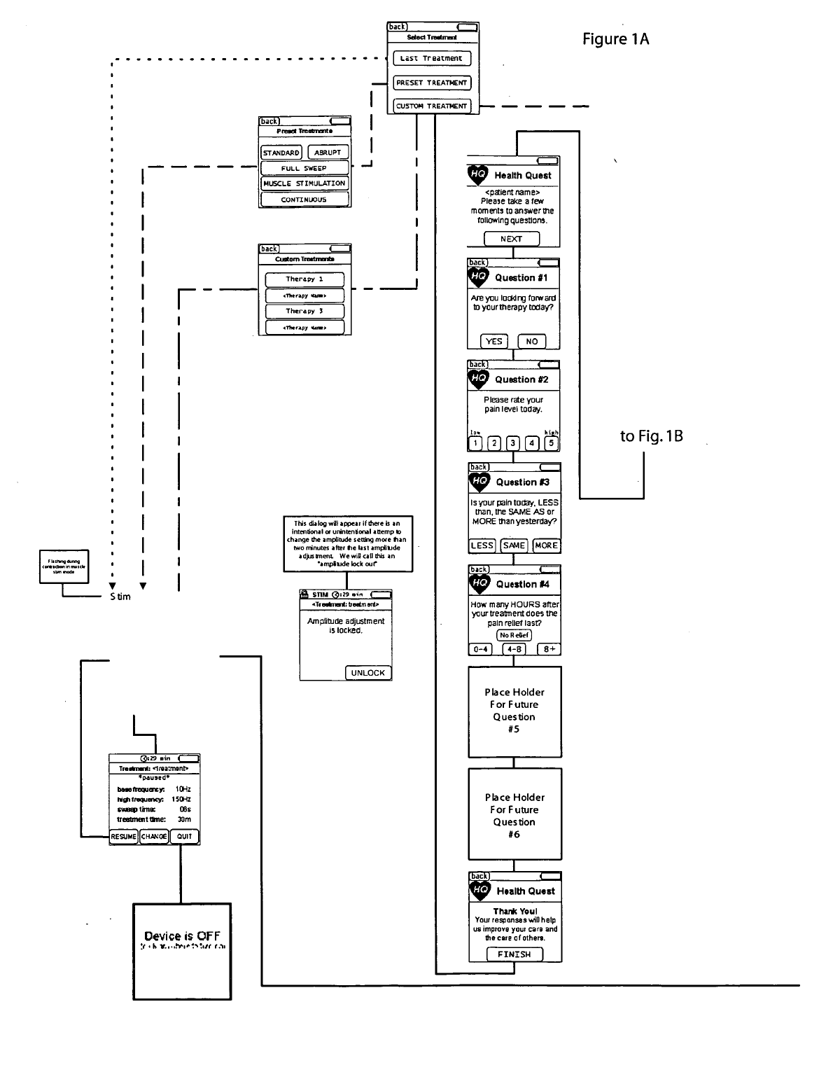

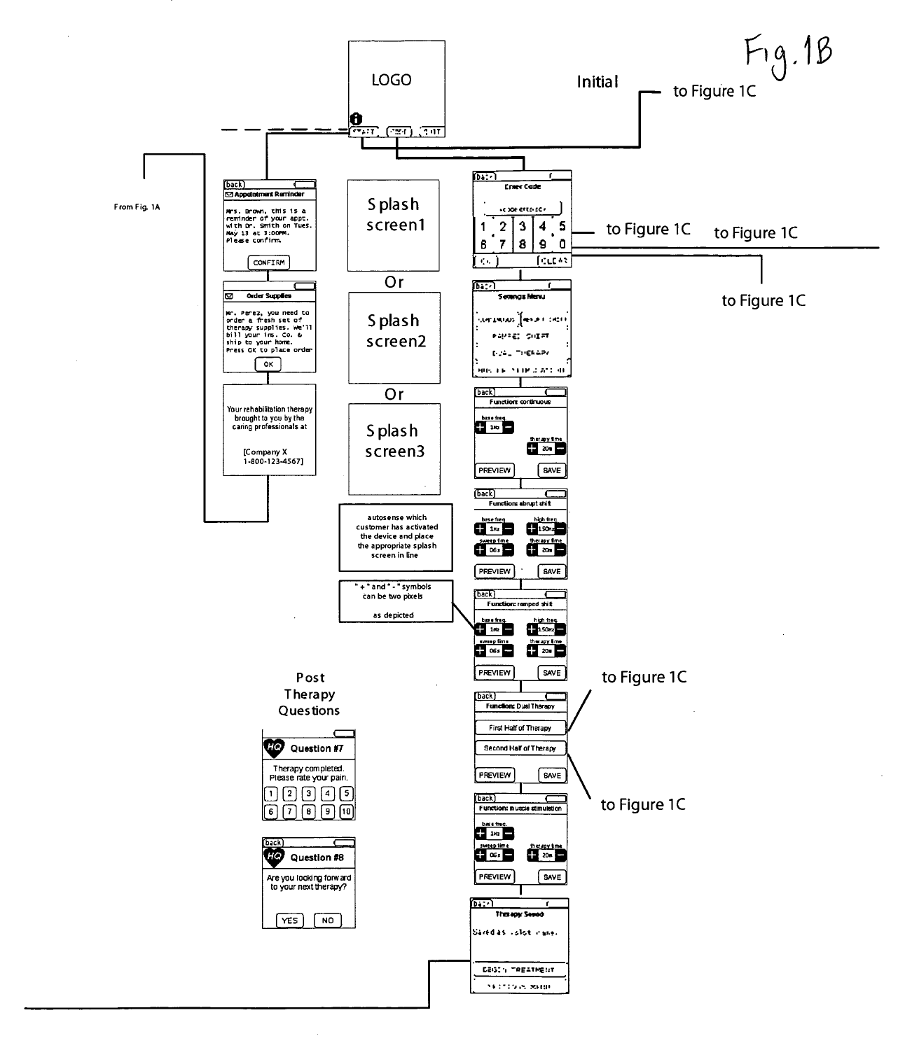

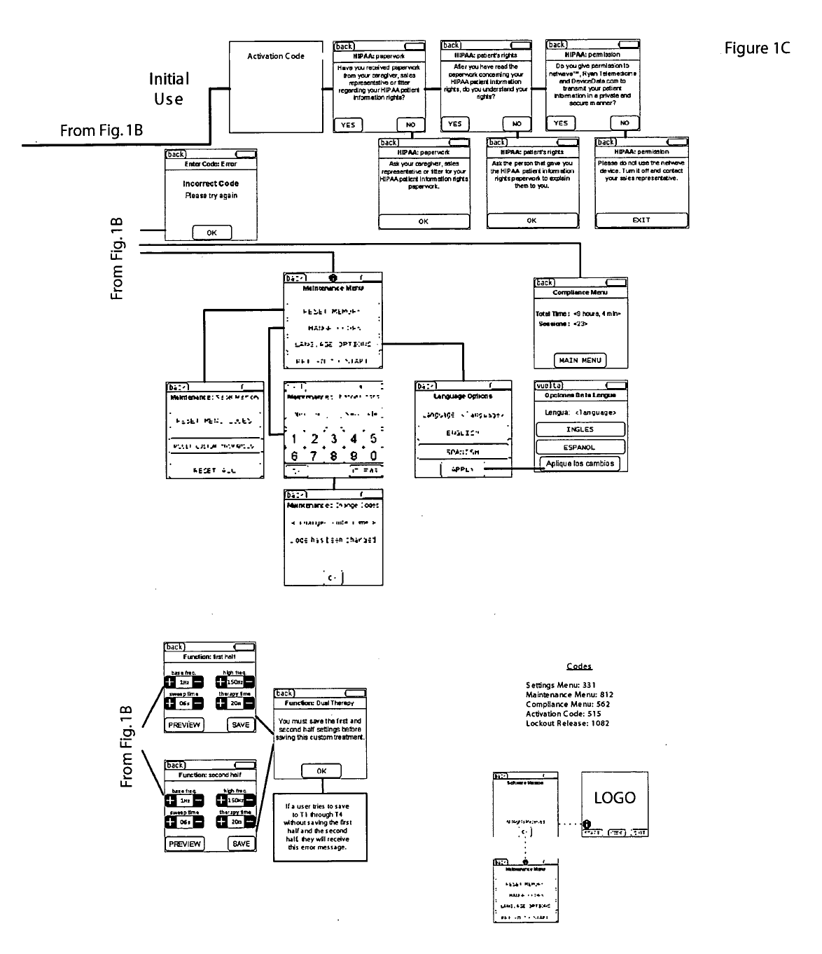

FIGS. 1A-1C represent an embodiment of the user interface. FIG. 1B comprises the Logo and/or splash screens that are activated upon startup. The user may be reminded of the appointment for treatment at that time if there was a previously scheduled treatment, or if the device is already on, a prompt may occur as an alarm to remind the user to perform treatment. The right portion of FIG. 1A then flows with questions that may be communicated to the treatment device remotely. The answers of these questions may be saved and/or transmitted back to the management component in order to assist in the treatment process. A treatment input is accepted and treatment occurs, leading to stimulation of the treatment area of the user. Maintenance and setup example menus are shown in FIG. 1C. As these menus are exemplary, one skilled in the art will recognize that any form of user interface that allows for measuring treatment, reporting treatment and accepting a modified treatment is in keeping with the spirit of the invention.

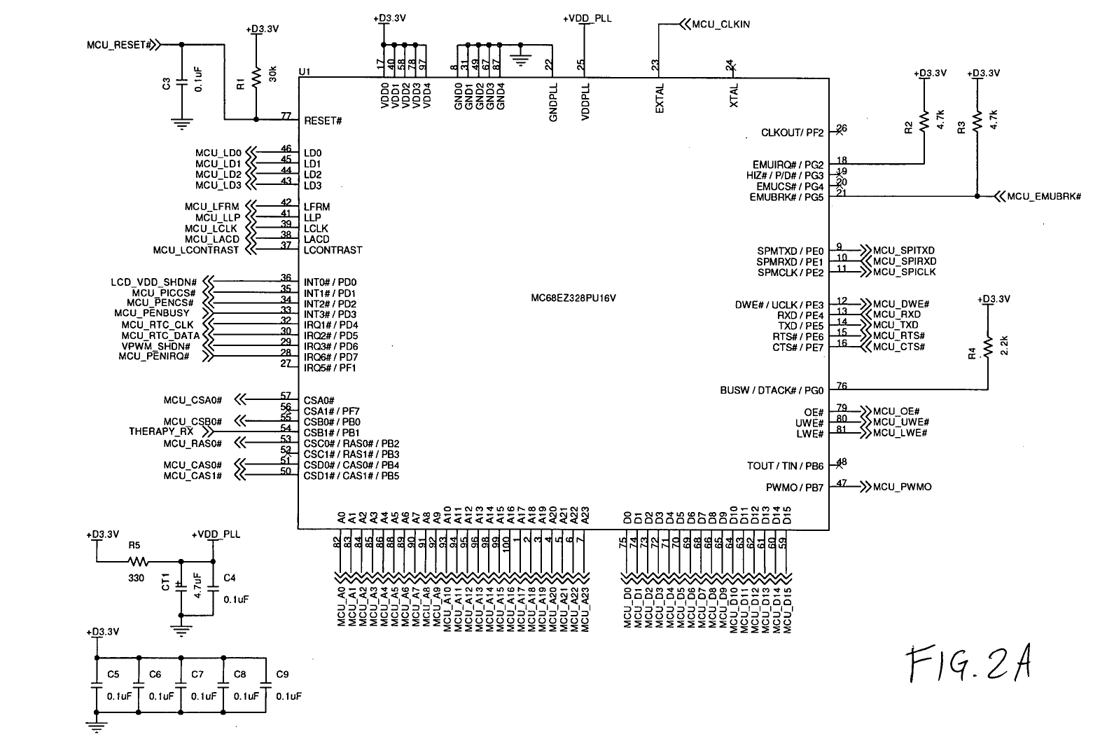

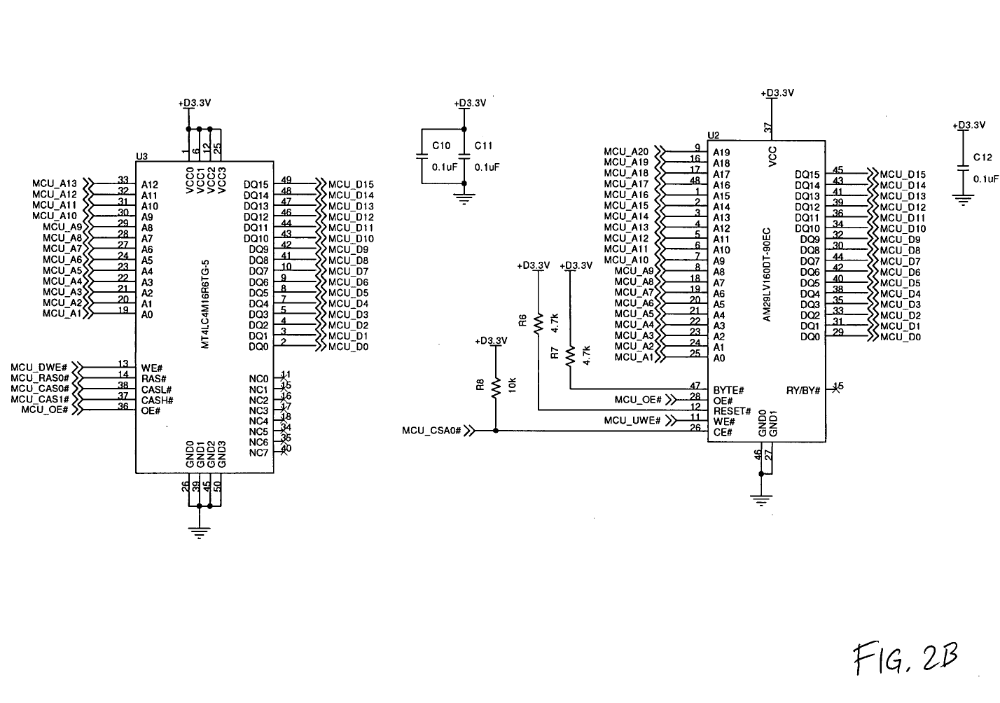

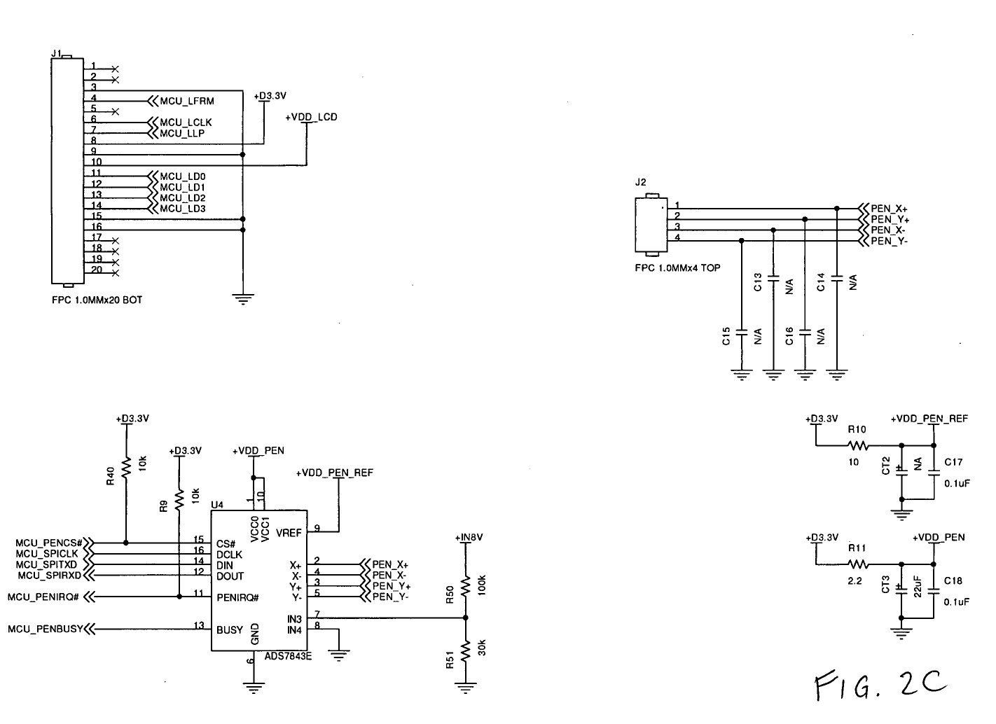

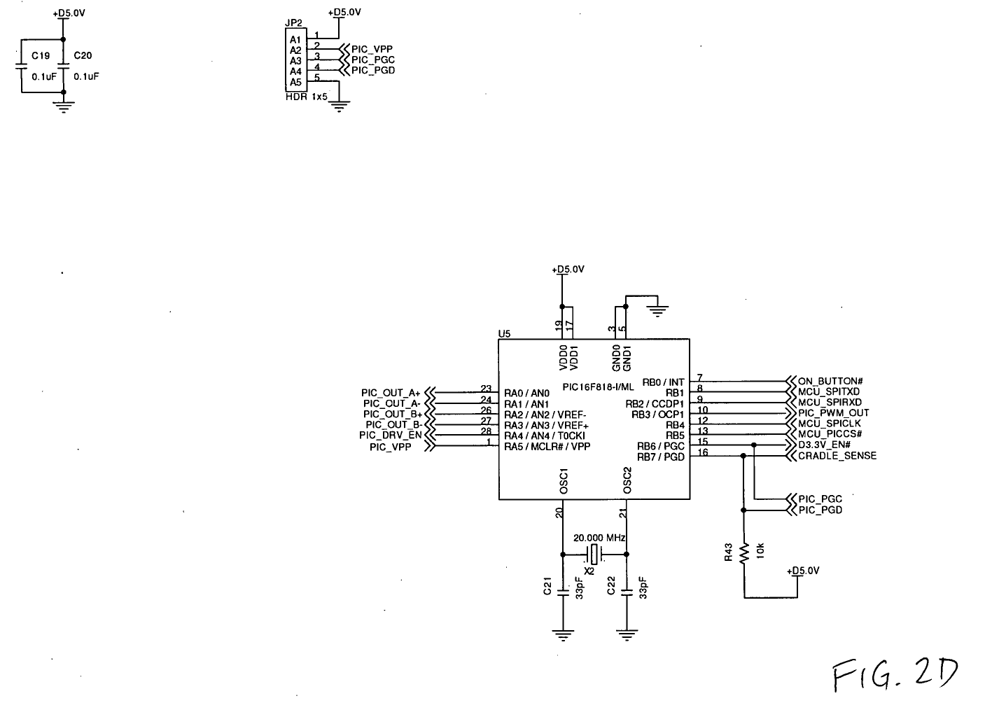

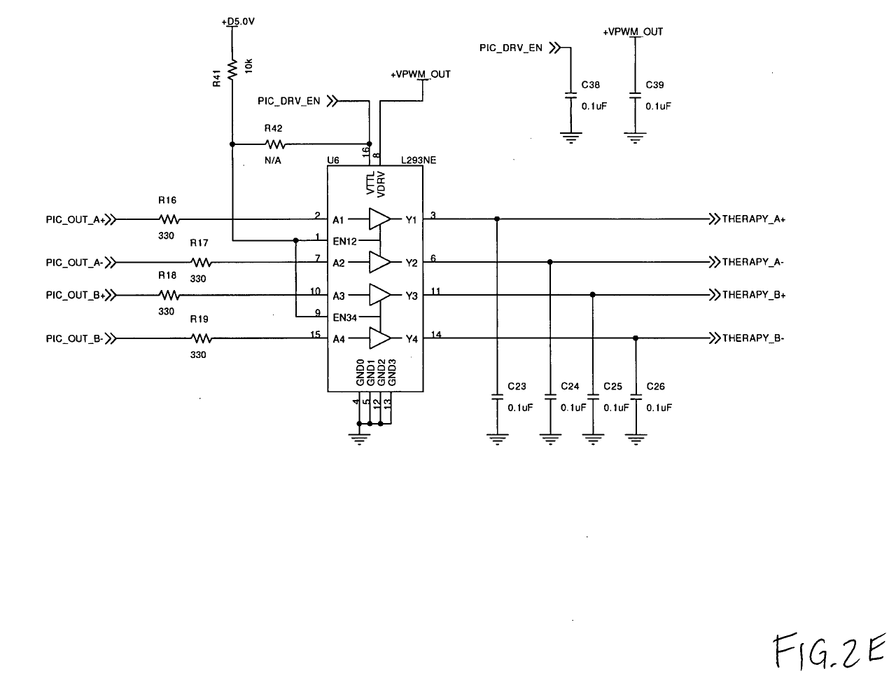

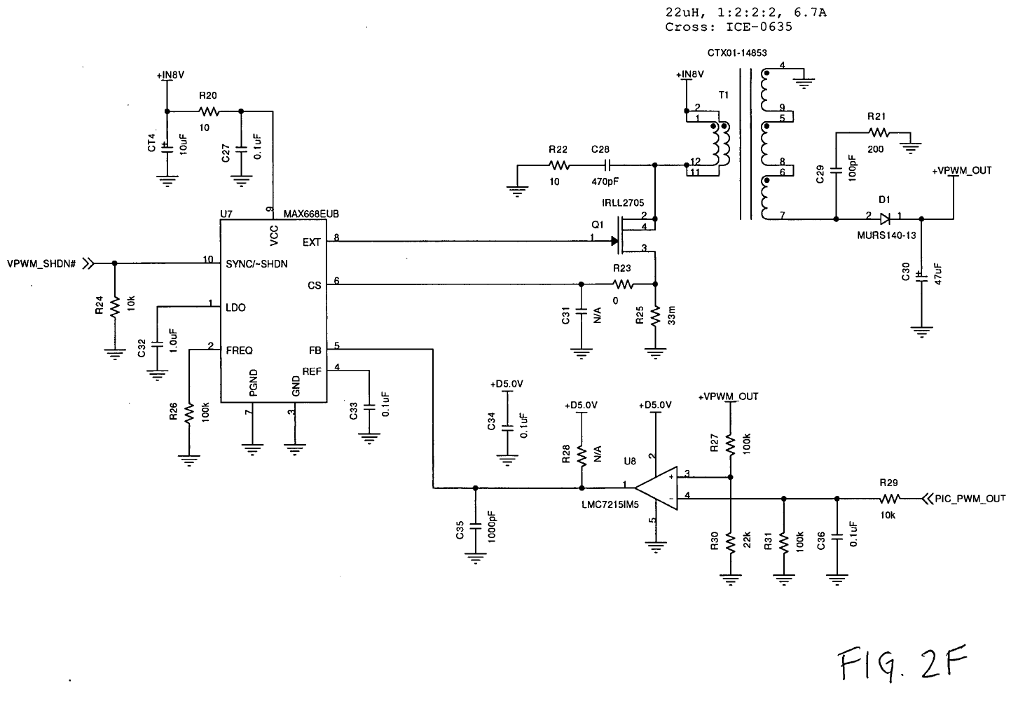

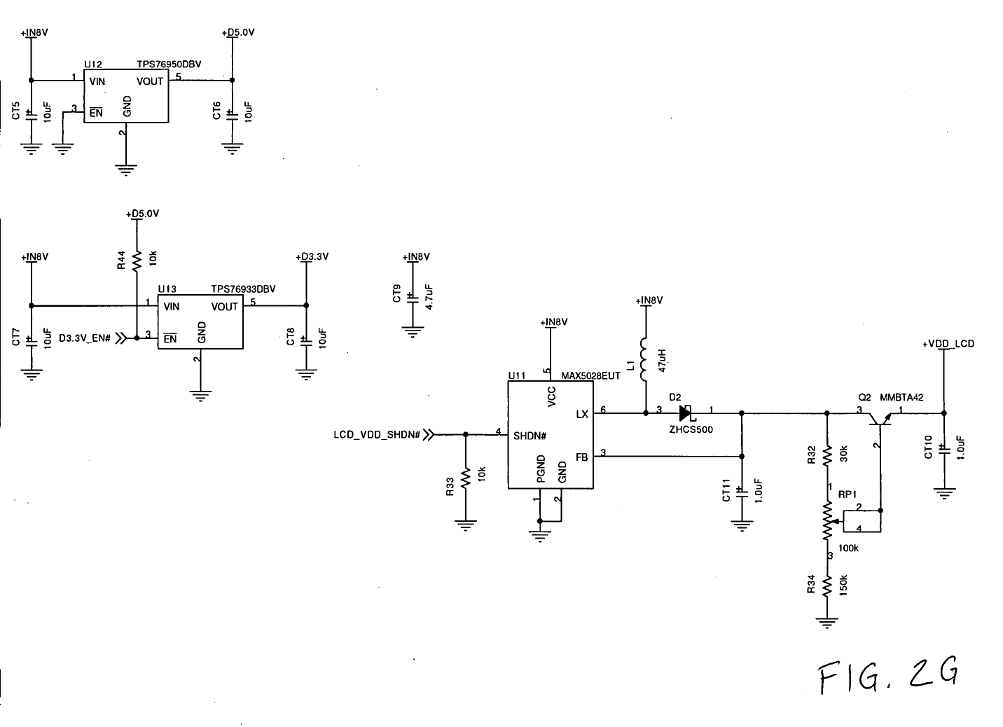

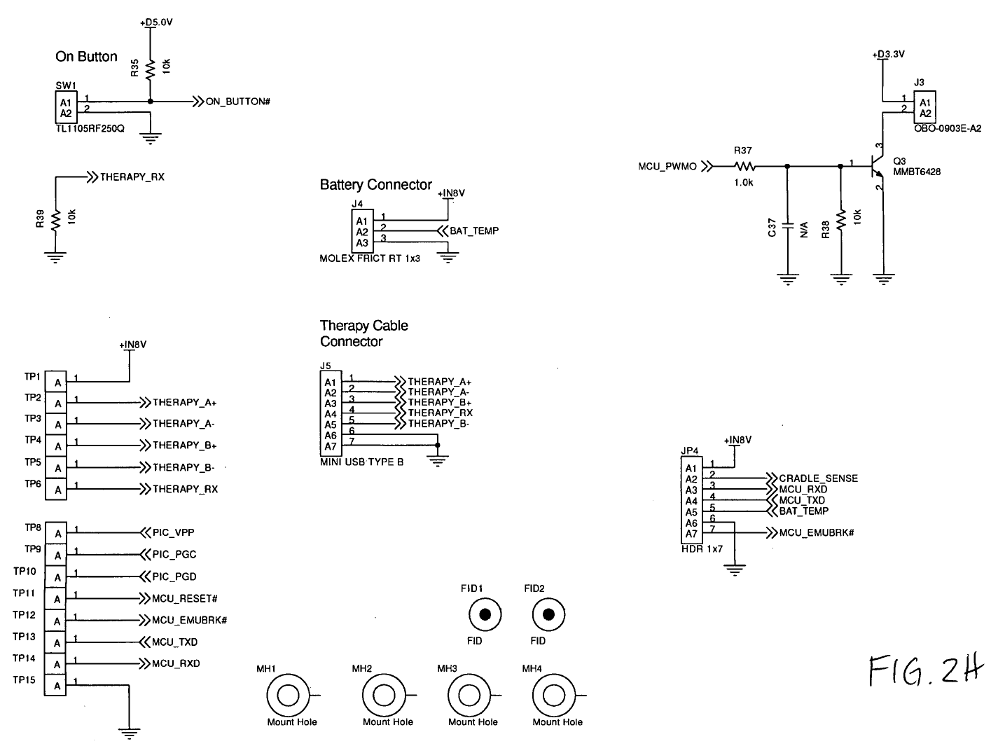

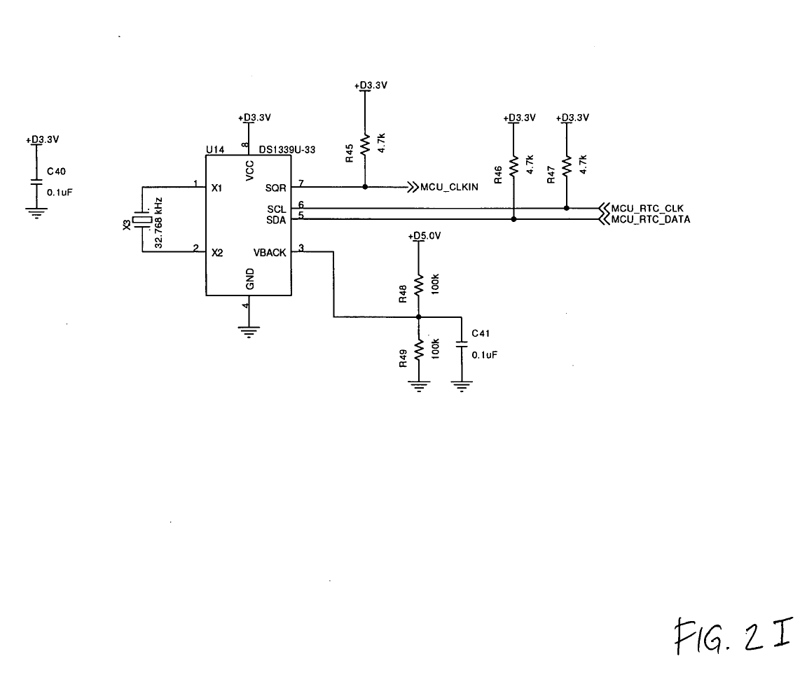

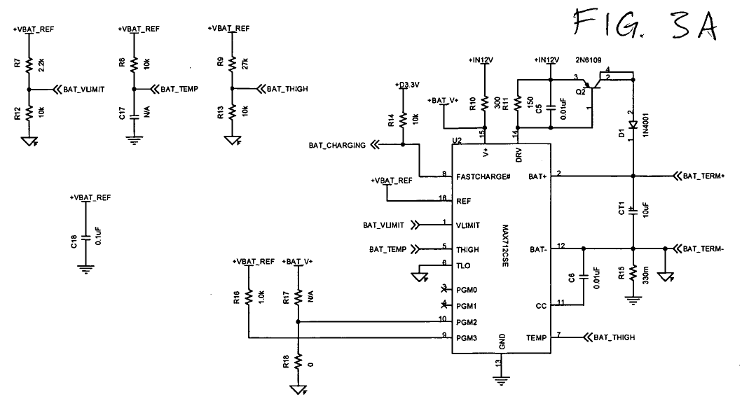

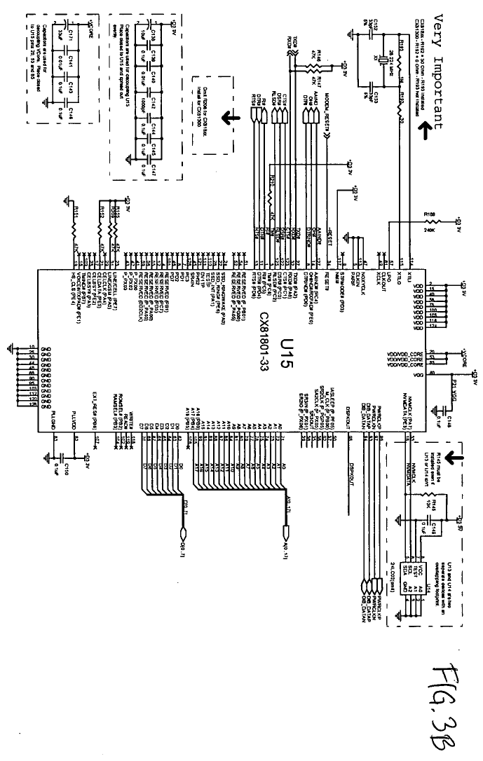

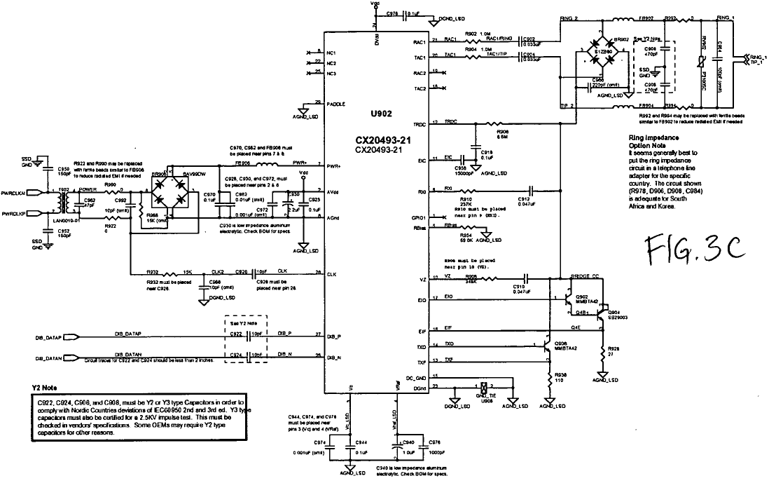

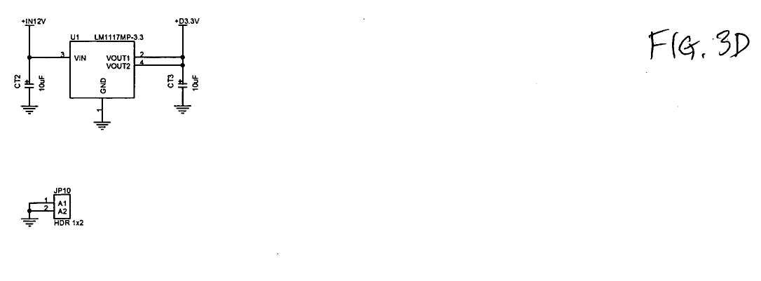

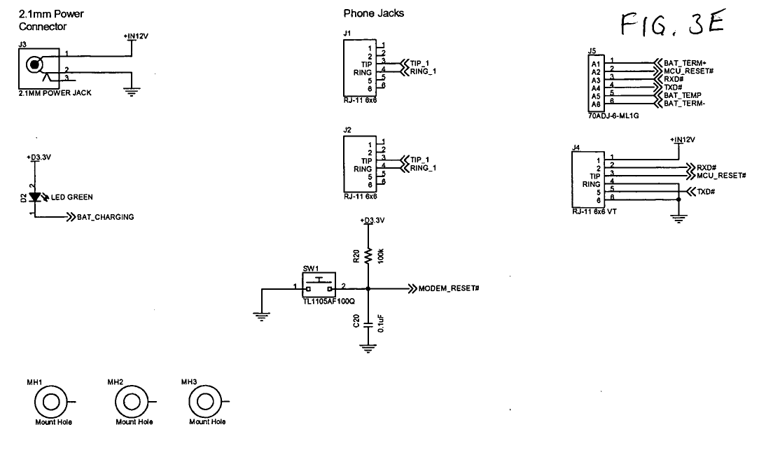

FIGS. 2A-2I show an embodiment of a hardware implementation for a treatment device configured to measure therapy, report treatment and obtain modified therapy over a communications component. FIG. 2A shows the processor “MC68EZ328” configured to drive an LED display, run a pulse width modulation controller and handle remote communications. Any other microprocessor may be used in place of this component. FIG. 2B shows DRAM component “MT4LC4M16R6TG” and flash memory component “AM29LV160DT”. Any other suitable memory devices may be used to hold therapy data and configuration parameters respectively. FIG. 2C shows various screen controller components. FIG. 2D shows a flash microcontroller. FIG. 2F shows PWM (pulse width modulation) controller “MAX668EUB”. All other components may be replaced with equivalent parts and modifications may be made to add wireless communications controllers or UARTs in order to allow for any type of remote communication so long as the device is still configured to measure therapy, report treatment and obtain modified therapy over a communications component. Adding a modem or other communication chip to the treatment device allows for a standalone version of the device, while configuring a docking station with a modem provides for a smaller treatment device but provides for remote communications to and from the treatment device and to and from a management module when the device is docked.

FIG. 3A-3E show an exemplary hardware implementation for a docking station. FIG. 3A shows fast charge controller “MAX712CSE” configured to charge the device specified in FIGS. 2A-2I. FIG. 3B shows a digital isolation barrier “CX20493”. FIG. 3F shows phone jacks that allow for hooking up the docking station to a phone line allowing for remote communications.

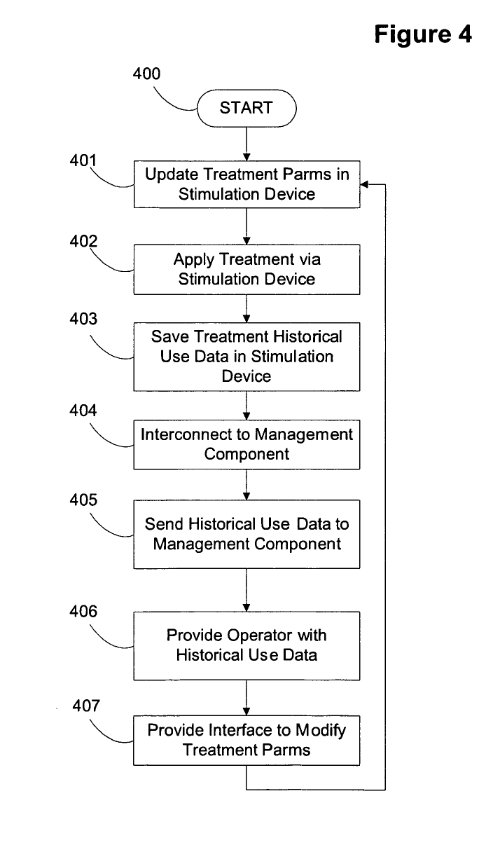

The interconnects between all devices shown in FIGS. 2A-21 and 3A-3E are marked and one skilled in the art will recognize that any deviations from these interconnects is in keeping with the spirit of the invention.

The communication component referenced herein may comprise a modem, computer network or any other interconnection fabric (wireless or wired) able to connect one or more of the devices with one or more of the management components. These communication components may be incorporated into the devices themselves, incorporated into the docking station or recharge unit, or part of a stand-alone component such as a cell phone or other communication device.

FIG. 4 is a flow chart illustrating an embodiment of a method for measuring, modifying and reporting treatment compliance. Processing begins at 400. When a decision is made to provide patients with a device for treatment the initial protocol or form of treatment can be entered by the medical professional responsible for oversight of the treatment. The stimulation device updates the treatment parameters at 401. The patient is then provided with a portable device that can be taken to a location of the patient's choosing for administration of the treatment protocol. The stimulation device provides treatment to the patient at 402. The treatment duration and patterns and any of the other parameters associated with treatment, such as the time of day or the number of treatments in a unit time period for example are saved at 403. The stimulation device connects with the management component using a communications component operating either wirelessly or using a docking station for example at 404. Using the communication component, the device periodically or regularly sends or reports the patients patterns of use and any desired historical use data back to the management component at 405. The management component provides the operator with the historical reports at 406. Based on these reports the operator of the management component can opt to modifying the therapy and/or simply store the reports to prove patient compliance with the treatment using an interfacing provided by the management component at 407.

The historical use data provides proof of treatment that enables the medical professional to know when a patient is non-compliant and then follow-up or take whatever next steps are required to attempt to achieve compliance. In some instance, the management component may, for instance, be configured to automatically provide the patient with a reminder (e.g., via email or phone) that a treatment was missed. The management component can be programmed to have various levels of non-compliance and take various actions at different levels. In the case of substantial non-compliance, for example, the system may report to a physician or insurance company who can then take whatever actions are desired (e.g., a phone call, letter, etc . . . ).

The historical use data collected from the devices also serves as a rich source for data mining to determine treatment effectiveness or derived other useful information. Although patient specific data must typically be removed to perform such analysis (particularly in the cases where the data or results of such analysis might be sold) mining the report data serves as a potential source of valuable information.

The management component may also contain an operator interface that enables a physician or other medical professional to review patient usage patterns and collect other information (e.g., vitals) in order to determine the effectiveness of the treatment regime. Based on the information obtained via this operator interface or based on the operators own personal knowledge, the operator may then modify the treatment protocol (e.g., by changing the duration or treatment, type of treatment or whatever other variables are considered pertinent). Upon receipt of an authorized modification command, the management component communication with the device via the communication component and initiates a change in the treatment protocol. Embodiments of the invention also contemplate using the communication component for messages and or data between the operator and patient.

Having the ability to set an initial treatment protocol (which can be varied by the patient within a range if such permission is granted by the medical professional responsible for treatment), remotely measure effectiveness and determine if the patient is complying with that treatment, and remotely modify the treatment based on reported treatment data provides a number of benefits in efficiency and thereby leads to increased revenue opportunities.

Insurance companies are sometimes reluctant to pay for claims relating to an ongoing treatment unless it is feasible to provide proof the treatment is truly ongoing. The device described herein, when combined with the management component provides a mechanism for not proving compliance with the treatment and can thereby lead to a higher profitability for those who receive payment from the insurance company. From the insurance companies perspective one or more aspects of the invention described herein provide a way to minimize fraud by guaranteeing that only compliant claims are paid. In instances where the patient is non-compliant, the physician is given a mechanism to seek further revenue by encouraging patient compliance.

One benefit of having a device and management component configured to communication is that it provides physicians with an efficient conduit to interact with their patient. In one or more embodiments of the invention, the management component is designed to interface with a patient's medical record and thereby given the physician enough information to conduct a remote patient consultation. For instance, when reviewing a patient's compliance report along with the patient's medical record the physician or other medical professional may use the conduit on the device to ask the patient questions and or obtain further information about the patient's vitals and/or general condition. As such the system described herein enables an efficient doctor patient visit to occur when both doctor and patient are in separate locations. In one or more embodiments of the invention the device is programmed upon receipt of correspondence from the operator to only initiate treatment once the patient has responded in the requested manner to the correspondence.

While the invention herein disclosed has been described by means of specific embodiments and applications thereof, numerous modifications and variations could be made thereto by those skilled in the art without departing from the scope of the invention set forth in the claims.

What is claimed is:

1. A system for measuring, modifying and reporting treatment compliance comprising:
an interferential stimulation device for administering a treatment wherein said interferential stimulation device is configured to obtain treatment parameters from an operator and wherein said interferential stimulation device is further configured provide treatment and save historical use data;
a communication component configured to periodically interconnect said interferential stimulation device with a management component;
said interferential stimulation device configured to send said historical use data associated with said treatment to said management component; and,
said management component configured to provide said historical use data to said operator and enable said operator to modify said treatment parameters in order to cause a modification in said treatment.

2. The system of claim 1 further comprising:
said management component further configured to report when said historical use data is non-compliant with a recommended treatment.

3. The system of claim 1 further comprising:
said management component further configured to send historical use data that is non-compliant with a recommended treatment to an insurance company.

4. The system of claim 1 further comprising:
said interferential stimulation device configured to obtain modified treatment parameters.

5. The system of claim 1 further comprising:
said interferential stimulation device configured to present a question to a patient wherein said question is obtained from said management component via said communication component.

6. The system of claim 1 further comprising:
said interferential stimulation device configured to send an answer from a patient wherein said answer is transmitted to said management component via said communication component.

7. The system of claim 1 further comprising:
said interferential stimulation device configured to present a question to a patient wherein said question is obtained from said management component via said communication component; and,
said interferential stimulation device configured to send an answer from a patient wherein said answer is transmitted to said management component via said communication component.

8. The system of claim 1 further comprising:
said interferential stimulation device configured to alert a patient of a missed treatment session.

9. The system of claim 1 further comprising:
said management component configured to alert a patient of a missed treatment session via a cell phone call or an email message.

10. The system of claim 1 further comprising:
said management component configured to mine data from a plurality of patients.

11. A method for measuring, modifying and reporting treatment compliance comprising:
updating treatment parameters in an interferential stimulation device for administering a treatment wherein said treatment parameters are obtained from an operator;
applying treatment;
saving historical use data in said interferential stimulation device;
interconnecting said interferential stimulation device periodically with a management component via a communication component;
sending historical use data from said interferential stimulation device associated with said treatment to said management component; and,
providing historical use data to said operator;
providing said operator with an interface to modify said treatment parameters in order to cause a modification in said treatment.

12. The method of claim 11 further comprising:
reporting from said management component when said historical use data is non-compliant with a recommended treatment.

13. The method of claim 11 further comprising:
sending from said management component historical use data that is non-compliant with a recommended treatment to an insurance company.

14. The method of claim 11 further comprising:
saving modified treatment parameters in said interferential stimulation device.

15. The method of claim 11 further comprising:
presenting a question to a patient via said interferential stimulation device wherein said question is obtained from said management component via said communication component.

16. The method of claim 11 further comprising:
sending an answer from a patent via said interferential stimulation device wherein said answer is transmitted to said management component via said communication component.

17. The method of claim 1 1 further comprising:
presenting a question to a patient via said interferential stimulation device wherein said question is obtained from said management component via said communication component; and,
sending an answer from a patent via said interferential stimulation device wherein said answer is transmitted to said management component via said communication component.

18. The method of claim 11 further comprising:
alerting a patient from said interferential stimulation device of a missed treatment session.

19. The method of claim 11 further comprising:
alerting a patient from said management component of a missed treatment session via a cell phone call or an email message.

20. The method of claim 11 further comprising:
mining data from a plurality of patients via said management component.
